(19) United States
(12) Patent Application Publication
(10) Pub. No.: US 2025/0076259 A1
(43) Pub. Date: Mar. 6, 2025

(54) ULTRASONIC PROBES AND ULTRASONIC DEVICES

(71) Applicant: WUHAN UNITED IMAGING HEALTHCARE CO., LTD., Wuhan (CN)

(72) Inventors: Changxing KE, Wuhan (CN); Hao HU, Wuhan (CN); Kang SI, Shanghai (CN)

(73) Assignee: WUHAN UNITED IMAGING HEALTHCARE CO., LTD., Wuhan (CN)

(21) Appl. No.: 18/817,320

(22) Filed: Aug. 28, 2024

(30) Foreign Application Priority Data
Aug. 29, 2023 (CN) 202311099779.2

(51) Int. Cl. G01N 29/24

(52) U.S. Cl. CPC G01N 29/2487; G01N 29/2443

(57) ABSTRACT

The present disclosure provides an ultrasonic probe and an ultrasonic device. The ultrasonic probe includes a backing part and a flexible circuit board. The backing part includes a curved top surface and a first side surface connected to one side of the top surface. The flexible circuit board includes a main board portion and a first connection portion. The main board portion is disposed on the top surface. One end of the first connection portion is connected with one side of the main board portion, and the other end of the first connection portion is configured to transmit an electrical signal. The first connection portion includes a plurality of branches. The plurality of branches of the first connection portion is disposed on the first side surface. The ultrasonic device includes the ultrasonic probe.

### CROSS REFERENCE TO RELATED APPLICATIONS

This application claims priority to Chinese Application No. 202311099779.2, filed on Aug. 29, 2023, the entire contents of which are incorporated herein by reference.

### TECHNICAL FIELD

The present disclosure relates to the field of ultrasonic technology, and in particular to an ultrasonic probe and an ultrasonic device.

### BACKGROUND

Flexible circuit boards are printed circuits made of polyester film or polyimide as substrates. The flexible circuit boards are widely used because of their high circuit density, light weight, and free bending and folding. The flexible circuit boards of ordinary linear array ultrasonic probes can be folded directly. For convex array probes, since the convex array structure has a specific curvature, the planar flexible circuit board needs to be folded into a curved flexible circuit board. When the planar flexible circuit board is folded along the arc edge of the convex array, a plurality of wrinkles may be generated. The wrinkles of the circuit board may occupy a larger space and increase the difficulty of the subsequent molding process. In addition, the wrinkles of the circuit board indicate that the circuits inside the circuit board are bent and deformed, and thus the wrinkles may reduce the reliability of the electrical connection inside the probe.

Therefore, it is desirable to provide an ultrasonic probe and an ultrasonic device that can solve the problems caused by folding of the flexible circuit board.

### SUMMARY

One or more embodiments of the present disclosure provide an ultrasonic probe, comprising a backing part and a flexible circuit board. The backing part may include a curved top surface and a first side surface connected to one side of the top surface. The flexible circuit board may include a main board portion and a first connection portion. The main board portion may be disposed on the top surface. One end of the first connection portion may be connected with one side of the main board portion, and the other end of the first connection portion may be configured to transmit an electrical signal. The first connection portion may include a plurality of branches. The plurality of branches of the first connection portion may be disposed on the first side surface.

In some embodiments, the backing part may further include a second side surface connected to the other side of the top surface. The flexible circuit board may include a second connection portion. One end of the second connection portion may be connected with the other side of the main board portion, the other end of the second connection portion may be configured to transmit the electrical signal. The second connection portion may include a plurality of branches. The plurality of branches of the second connection portion may be disposed on the second side surface.

In some embodiments, the first side surface and/or the second side surface may be outward convexly curved or inward concavely curved.

In some embodiments, the backing part may further include a third side surface and a fourth side surface. The first side surface may be opposite to the second side surface. The third side surface may be opposite to the fourth side surface. The flexible circuit board may further include a third connection portion and a fourth connection portion.

In some embodiments, a first count of the plurality of branches of the first connection portion and a second count of the plurality of branches of the second connection portion may be the same or different. A first spacing distance between two adjacent branches of the plurality of branches of the first connection portion and a second spacing distance between two adjacent branches of the plurality of branches of the second connection portion may be the same or different.

In some embodiments, the two adjacent branches of the plurality of branches of the first connection portion may be spaced by a first spacing region.

In some embodiments, the first spacing region may include a first hollow region; or the first spacing region may include a first flexible region. A rigidity of the first flexible region may be less than a rigidity of each of the plurality of branches of the first connection portion.

In some embodiments, the first flexible region may be elastic.

In some embodiments, at least one end of the first spacing region may be provided with an arced chamfer.

In some embodiments, the ultrasonic probe may further comprise a piezoelectric layer. The flexible circuit board may be disposed between the piezoelectric layer and the backing part. The flexible circuit board may be configured to transmit a signal from the piezoelectric layer. The piezoelectric layer may include a plurality of crystal units arranged in an array. The first spacing distance between the two adjacent branches of the plurality of branches of the first connection portion may be positively correlated with a third spacing distance between two adjacent crystal units of the plurality of crystal units.

In some embodiments, the flexible circuit board may include a first connection structure. The other end of the first connection portion may be connected with the first connection structure.

In some embodiments, the backing part may further include a bottom surface. The first side surface may be connected between the top surface and the bottom surface. The first connection structure may include a secondary board portion and an adapter portion. The first connection portion may be connected between the main board portion and the secondary board portion. The secondary board portion may be connected between the first connection portion and the adapter portion. The secondary board portion may be disposed on the bottom surface.

In some embodiments, the secondary board portion may include a plurality of branches.

In some embodiments, two adjacent branches of the plurality of branches of the secondary board portion may be spaced by a second spacing region.

In some embodiments, the second spacing region may include a second hollow region; or the second spacing region may include a second flexible region. A rigidity of the second flexible region may be less than a rigidity of each of the plurality of branches of the secondary board portion.

In some embodiments, the second flexible region may be elastic.

In some embodiments, the first connection portion and the first connection structure may form an adapter board. A top end of the adapter board may be connected with the main board portion. The ultrasonic probe may include at least two adapter boards.

In some embodiments, the top end of the adapter board may be curved.

In some embodiments, the bottom surface of the backing part may be curved, and the bottom surface of the backing part may be provided with a heat dissipation structure.

One or more embodiments of the present disclosure provide an ultrasound device, comprising an ultrasonic probe. The ultrasonic probe may include a backing part and a flexible circuit board. The backing part may include a curved top surface and a first side surface connected to one side of the top surface. The flexible circuit board may include a main board portion and a first connection portion. The main board portion may be disposed on the top surface. One end of the first connection portion may be connected with one side of the main board portion, and the other end of the first connection portion may be configured to transmit an electrical signal. The first connection portion may include a plurality of branches. The plurality of branches of the first connection portion may be disposed on the first side surface.

The beneficial effects of the embodiments of the present disclosure include but are not limited to the following descriptions. By providing the flexible circuit board, the ultrasonic probe of some embodiments of the present disclosure does not require adapter, and has high reliability; the problem of wrinkles generated a convex array surface by folding a planar flexible circuit board can be effectively solved, and the influence of the wrinkles on the reliability of electrical connection can be avoided; in addition, the assembly operations of the circuit board can be simplified, and assembly is easy.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering indicates the same structure, wherein:

FIG. 1 is a structural diagram illustrating an exemplary ultrasonic probe according to some embodiments of the present disclosure;

FIG. 2 is a structural diagram illustrating an exemplary ultrasonic probe according to some embodiments of the present disclosure;

FIG. 3 is a structural diagram illustrating an exemplary ultrasonic probe according to some embodiments of the present disclosure;

FIG. 4 is a structural diagram illustrating an exemplary flexible circuit board according to some embodiments of the present disclosure;

FIG. 5 is a structural diagram illustrating an exemplary flexible circuit board according to some embodiments of the present disclosure;

FIG. 6 is a structural diagram illustrating an exemplary ultrasonic probe according to some embodiments of the present disclosure;

FIG. 7 is a structural diagram illustrating an exemplary backing part according to some embodiments of the present disclosure;

FIG. 8 is a cross-sectional view illustrating an exemplary backing part according to some embodiments of the present disclosure;

FIG. 9 is a cross-sectional view illustrating an exemplary backing part according to some embodiments of the present disclosure;

FIG. 10 is a cross-sectional view illustrating an exemplary backing part according to some embodiments of the present disclosure;

FIG. 11 is a cross-sectional view illustrating an exemplary backing part according to some embodiments of the present disclosure;

FIG. 12 is a structural diagram illustrating an exemplary ultrasonic probe according to some embodiments of the present disclosure;

FIG. 13 is a structural diagram illustrating an exemplary flexible circuit board according to some embodiments of the present disclosure;

FIG. 14 is a structural diagram illustrating an exemplary ultrasonic probe according to some embodiments of the present disclosure;

FIG. 15 is a structural diagram illustrating an exemplary adapter board according to some embodiments of the present disclosure;

FIG. 16 is a structural diagram illustrating an exemplary ultrasonic probe according to some embodiments of the present disclosure;

FIG. 17 is a structural diagram illustrating an exemplary connection between a piezoelectric layer and a flexible circuit board and a backing part according to some embodiments of the present disclosure; and

FIG. 18 is a structural diagram illustrating an exemplary piezoelectric layer according to some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person having ordinary skills in the art to apply the present disclosure to other similar scenarios in accordance with these drawings without creative labor. Unless obviously obtained from the context or the context illustrates otherwise, the same numeral in the drawings refers to the same structure or operation.

A flexible circuit board may be configured to transmit electrical signals between a host and piezoelectric crystals of an ultrasonic device. A convex array probe is a probe with a curved surface on which array elements are arranged in a curved manner. The host may be mainly configured to process and display the signals received from the ultrasonic probe.

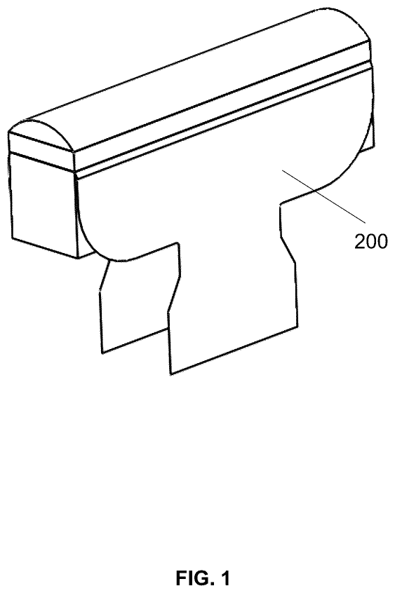

FIG. 1 is a structural diagram illustrating an exemplary ultrasonic probe according to some embodiments of the present disclosure. In some embodiments, as shown in FIG. 1, a flexible circuit board 200 of a linear array ultrasonic probe may be folded down directly.

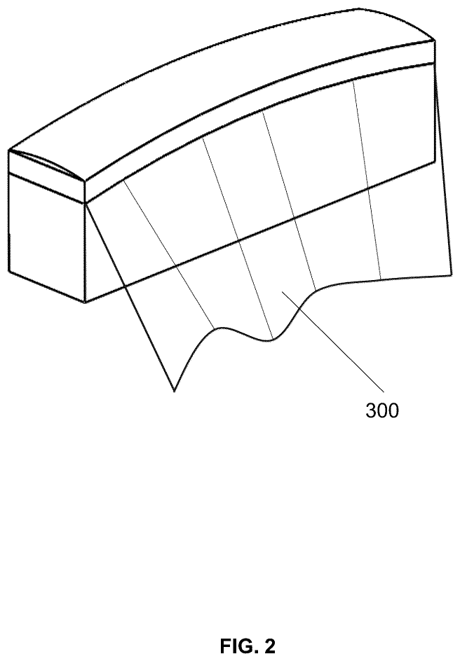

FIG. 2 is a structural diagram illustrating an exemplary ultrasonic probe according to some embodiments of the present disclosure. In some embodiments, as shown in FIG. 2, for a convex array probe, since a convex array structure has a specific curvature, a planar flexible circuit board 300 may be folded into a curved flexible circuit board 300. When the planar flexible circuit board 300 is folded down along a curved edge of the convex array structure, a plurality of wrinkles may be generated, affecting the reliability of an internal electrical connection of the ultrasonic probe and increasing the difficulty of the subsequent molding process in production.

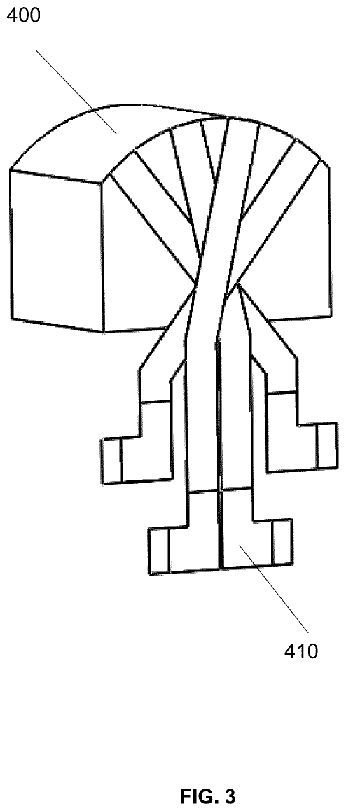

FIG. 3 is a structural diagram illustrating an exemplary ultrasonic probe according to some embodiments of the present disclosure. In some embodiments, as shown in FIG. 3, a plurality of individual pins 410 may be led out from an edge of a flexible circuit board 400 of the ultrasonic probe, and then folded into a plane to form a convex array. Since each of the plurality of individual pins 410 of the flexible circuit board 400 is separated independently, a surface tension generated when the flexible circuit board 400 is folded may be weakened, and the problem of wrinkles generated in the flexible circuit board 400 may be solved. However, the flexible circuit board 400 of the solution may have a complex structural design, low space utilization, and require the use of more connectors, which is cumbersome to be assembled and has poor reliability.

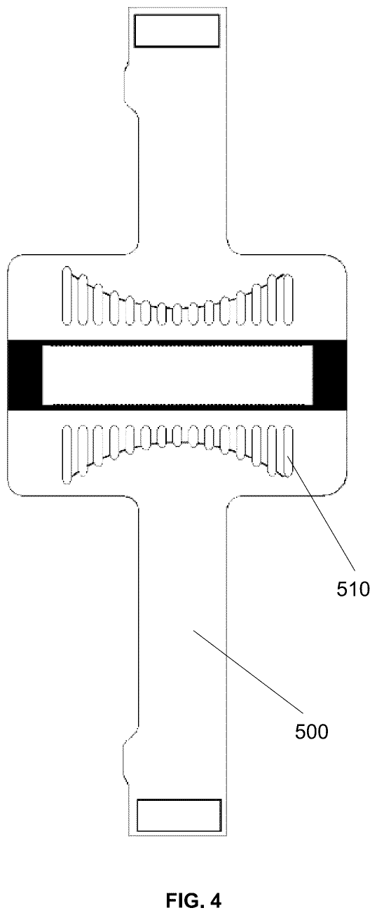

FIG. 4 is a structural diagram illustrating an exemplary flexible circuit board according to some embodiments of the present disclosure. In some embodiments, as shown in FIG. 4, hollow portions 510 may be disposed on both sides of a main board of a flexible circuit board 500. Outer edges of the hollow portions 510 may be set to be curves to match a curvature of a convex array structure. The flexible circuit board 500 may be bent along the curves. However, the hollow portions 510 of the solution may be arched during a bending process, causing lead wires in the flexible circuit board 500 to overlap and affecting the imaging quality.

To this end, some embodiments of the present disclosure provide an ultrasonic probe which solves the problem of the wrinkles caused by folding the circuit board by setting a structure of a flexible circuit board for the convex array structure, improving the reliability of the electrical connection inside the flexible circuit board, and reducing the difficulty of the subsequent process.

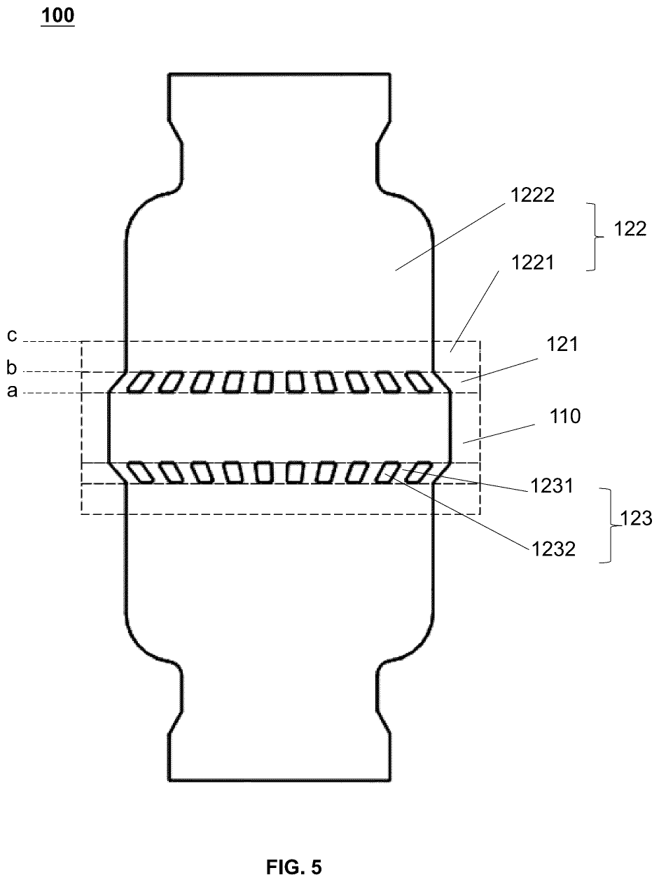

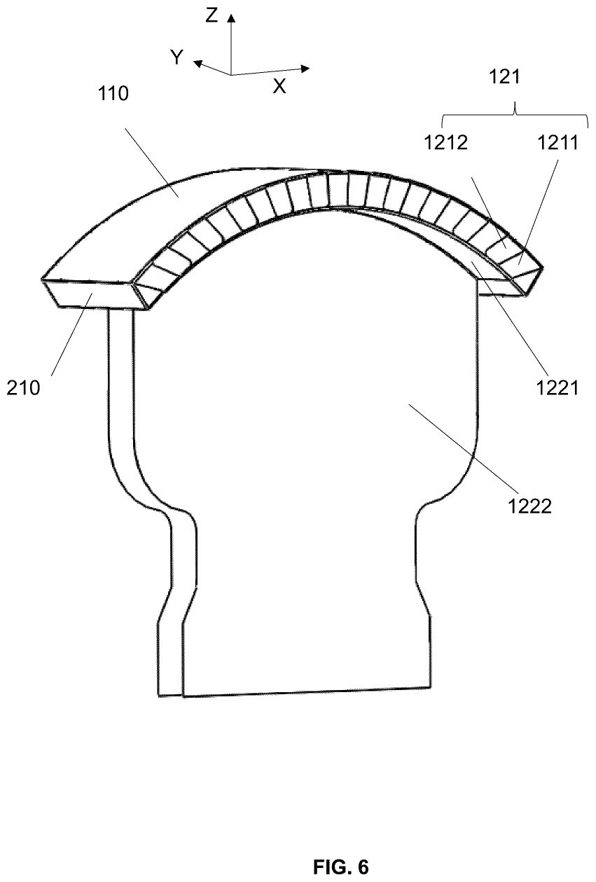

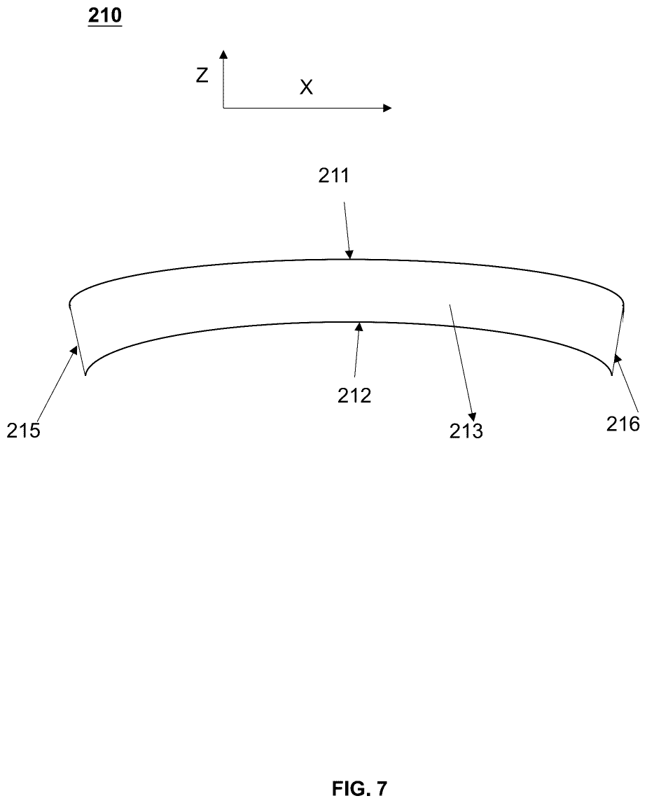

FIG. 5 is a structural diagram illustrating an exemplary flexible circuit board according to some embodiments of the present disclosure. FIG. 6 is a structural diagram illustrating an exemplary ultrasonic probe according to some embodiments of the present disclosure. FIG. 7 is a front view illustrating an exemplary backing part according to some embodiments of the present disclosure. The front view may be considered as a view of the backing part on an X-Z plane.

In some embodiments, as shown in FIGS. 5-6, the ultrasonic probe may include a backing part 210 and a flexible circuit board 100. As shown in FIG. 7, the backing part 210 may include a curved top surface 211 and a first side surface 213 connected to one side of the top surface 211. The flexible circuit board 100 may include a main board portion 110 and a first connection portion 121. The main board portion 110 may be disposed on the curved top surface 211 of the backing part 210. One end of the first connection portion 121 may be connected with one side of the main board portion 110, and the other end of the first connection portion 121 may be configured to transmit an electrical signal. The first connection portion 121 may include a plurality of branches 1211. The plurality of branches 1211 of the first connection portion 121 may be disposed on the first side surface 213 of the backing part 210.

The backing part 210 refers to a sound absorbing block connected to a back surface of a piezoelectric layer included in the ultrasonic probe. The backing part 210 may be made of a mixture of thermoplastic resin, tungsten powder, etc. The backing part 210 may be configured to absorb adverse sound waves radiated from the back of the piezoelectric layer to improve the signal quality of the ultrasonic probe, and may also serve as a structural support block to ensure the reliability of an array element structure of the ultrasonic probe.

The top surface 211 of the backing part 210 may form a curved surface protruding in a direction away from an interior of the top surface 211. In some embodiments, the top surface 211 of the backing part 210 may be a partial cylindrical surface. In some embodiments, the first side surface 213 of the backing part 210 may be connected with an edge of the top surface 211. The first side surface 213 may be a plane or a curved surface with a certain curvature. The first side surface 213 may protrude in a direction away from an interior of the first side surface 213 or recess in a direction close to the interior of the first side surface 213. In some embodiments, if the top surface 211 is a cylindrical surface, a generatrix of the cylindrical surface may be at a certain angle to the first side surface 213, such as 90° or any other angle. The generatrix refers to any edge on a cylindrical side parallel to a central axis. In some embodiments, the top surface 211 and the first side surface 213 may be smoothly connected. In some embodiments, the top surface 211 and the first side surface 213 may be unsmoothly connected, and a connection portion between the top surface 211 and the first side surface 213 may include a ridge. In some embodiments, the backing part 210 may further include a bottom surface 212 and a second side surface. More descriptions regarding the bottom surface 212 and the second side surface may be found in the following descriptions.

The flexible circuit board 100 refers to a printed circuit made of a flexible material (e.g., a polyester film or polyimide) as a substrate. The flexible circuit board 100 may have a high line density and low weight, and may be bent and folded freely. In some embodiments, the flexible circuit board 100 may be partially disposed on the backing part 210 to be supported by the backing part 210, thereby improving the stability of the device and making full use of an internal space of the ultrasonic probe. In some embodiments, the flexible circuit board 100 may be an integrated structure formed in one piece.

In some embodiments, the backing part 210 and the flexible circuit board 100 may be connected by bonding. For example, the backing part 210 and the flexible circuit board 100 may be bonded using an adhesive substance (e.g. glue) such that the backing part 210 and the flexible circuit board 100 may fit well. In some embodiments, if the backing part 210 and the flexible circuit board 100 match in shape and achieve good fit, no adhesive substance may be used for bonding the backing part 210 and the flexible circuit board 100. In some embodiments, some portions (e.g., portions with good shape matching) between the backing part 210 and the flexible circuit board 100 may not be bonded with the adhesive substance, while other portions between the backing part 210 and the flexible circuit board 100 may be bonded with the adhesive substance such that the backing part 210 and the flexible circuit board 100 may fit well.

In some embodiments, the main board portion 110 and the first connection portion 121 may be integrated to form the flexible circuit board 100.

The main board portion 110 may be a main component of the flexible circuit board 100. In some embodiments, the main board portion 110 may be provided with a main circuit of the flexible circuit board 100 to implement main functions (e.g., supplying power to the piezoelectric layer, etc.) of the flexible circuit board 100.

In some embodiments, a length L of the main board portion 110 may be determined by parameters of the ultrasonic probe. For example, the length L of the main board portion 110 may be calculated according to the following Equation (1):

$$L \geq d ⋆ N , \qquad ( 1 )$$

wherein, d denotes a distance between centers of adjacent array elements inside the ultrasonic probe, and N denotes a count of the array elements. In some embodiments, the piezoelectric crystals disposed in the ultrasonic probe may be evenly cut into a plurality of parts. Each (referred to as an array element) of the plurality of parts may be a smallest unit capable of independently transmitting and receiving ultrasonic waves.

In some embodiments, the main board portion 110 may be disposed on the curved top surface 211 of the backing part 210 such that the main board portion 110 may be closely attached to the piezoelectric layer disposed on the backing part 210, which realizes a compact structure, saves space, and achieves better signal transmission. In some embodiments, if the ultrasonic probe is a convex array probe, the main board portion 110 may be disposed on the top surface 211 of the backing part 210 based on a curvature of the convex array structure.

In some embodiments, a shape of the main board portion 110 may not be limited and may be set according to the needs of the ultrasonic probe. It the shape of the main board portion 110 may be a regular shape (e.g., a rectangle, a polygon, etc.) or an irregular shape.

The first connection portion 121 may be configured to be connected with the main board portion 110 and transmit the electrical signal. In some embodiments, when the ultrasonic probe is not assembled, the first connection portion 121 and the main board portion 110 may be disposed in the same plane; after the ultrasonic probe is assembled, the first connection portion 121 and the main board portion 110 may not be disposed in the same plane. In some embodiments, the first connection portion 121 may be disposed on the first side surface 213 of the backing part 210.

In some embodiments, a connection between the first connection portion 121 and the main board portion 110 may be configured as a curved transition connection to avoid stress concentration and reduce or eliminate the problem of line breakage that may occur after bending.

In some embodiments, the first connection portion 121 may include a plurality of branches 1211. In some embodiments, both ends of each of the plurality of branches 1211 may have different widths. For example, a width of one end of each of the plurality of branches 1211 connected with the main board portion 110 may be greater than a width of the other end of each of the plurality of branches 1211. In some embodiments, a sum of the width of one end of each of the plurality of branches 1211 connected with the main board portion 110 may be greater than a sum of the width of the other end of each of the plurality of branches 1211.

In some embodiments, shapes of the plurality of branches 1211 may be the same or different. In some embodiments, sizes (e.g., lengths, widths, surface areas, etc.) of the plurality of branches 1211 may be the same or different.

In some embodiments, two adjacent branches 1211 of the plurality of branches 1211 of the first connection portion 121 may be spaced by a first spacing region 1212. In some embodiments, the two adjacent branches 1211 of the plurality of branches 1211 of the first connection portion 121 may not be spaced by the first spacing region 1212. For example, side edges of the two adjacent branches 1211 of the plurality of branches 1211 may contact each other.

In some embodiments, the first spacing region 1212 may include a first hollow region. In some embodiments, the first hollow region may be a hollow through hole disposed in the flexible circuit board 100. The first hollow region may have one of various regular or irregular shapes, such as a rectangle, an ellipse, etc. The first hollow region may reduce wrinkles caused by the main board portion disposed on the curved top surface of the backing part.

In some embodiments, an upper edge and a lower edge of the first hollow region may correspond to an edge of the top surface 211 and an edge of the bottom surface 212 of the backing part 210, respectively. In this case, one end of each of the plurality of branches 1211 of the first connection portion 121 may be connected with the main board portion 110, and the other end of each of the plurality of branches 1211 of the first connection portion 121 may transmit the electrical signal (e.g., the other end of each of the plurality of branches 1211 of the first connection portion 121 may be connected with a first connection structure 122 described below). In some embodiments, the upper edge and the lower edge of the first hollow region may not correspond to the edge of the top surface 211 and the edge of the bottom surface 212 of the backing part 210, respectively. For example, the upper edge and the lower edge of the first hollow region may both be disposed on the first side surface 213 of the backing part 210. As another example, at least one of the upper edge and the lower edge of the first hollow region may exceed the edge of the top surface 211 and/or the edge of the bottom surface 212 of the backing part 210. In this case, the plurality of branches 1211 of the first connection portion 121 may be partially disposed on the top surface 211 and/or the bottom surface 212.

In some embodiments, at least one end of the first spacing region 1212 may be provided an arced chamfer. In some embodiments, at least one end of the first hollow region may be provided an arced chamfer. In some embodiments, both ends of the first hollow region may be provided an arced chamfer, respectively. By setting the arced chamfer, the stress concentration after the flexible circuit board is bent can be reduced, and breakage of the flexible circuit board during use can be prevented.

In some embodiments, the first spacing region 1212 may include a first flexible region. The first flexible region refers to a region in the flexible circuit board 100 that is more flexible than other regions. In some embodiments, a rigidity of the first flexible region may be less than a rigidity of each of the plurality of branches 1211 of the first connection portion 121. In some embodiments, the first flexible region may be made of a material that is more flexible than the plurality of branches 1211.

In some embodiments of the present disclosure, adjacent branches of the plurality of branches 1211 may be connected by a more flexible material, which does not affect the circuit performance, the wrinkles are not generated when the first connection portion is fit to the first side surface of the backing part, and the process operation is simpler.

In some embodiments, the first flexible region may be elastic. By providing an elastic region between the adjacent branches of the plurality of branches 1211, the backing part and the first connection portion can be allowed to have a certain size change or error to a certain extent, further improving the fit degree between the first connection portion and the backing part.

In some embodiments, if the ultrasonic probe is a convex array probe, the plurality of branches 1211 may be distributed in a curved shape, i.e., lower edges of the plurality of branches 1211 may be arranged in the curved shape. In some embodiments, an arrangement of the plurality of branches 1211 may be uniform or non-uniform. For example, the plurality of branches 1211 may be randomly arranged, and sizes (e.g., areas of the first hollow region) of the first spacing region 1212 between the adjacent branches 1211 of the plurality of branches 1211 may be different. In some embodiments, the edge of the top surface 211 of the backing part 210 may not exceed upper edges of the plurality of branches 1211, and an edge of the bottom surface 212 of the backing part 210 may not exceed the lower edges of the plurality of branches 1211. In some embodiments, lengths of the plurality of branches 1211 may be the same as a thickness of the backing part 210 on which the plurality of branches 1211 are disposed, i.e., the upper edges and the lower edges of the plurality of branches may correspond to the edge of the top surface 211 and the edge of the bottom surface 212 of the backing part 210, respectively, such that the plurality of branches 1211 may completely cover the first side surface 213. In some embodiments, the length of each of the plurality of branches may exceed the thickness of the backing part 210 on which the plurality of branches 1211 are disposed, i.e., the upper edges of the plurality of branches 1211 may exceed the edge of the top surface 211 of the backing part 210, and/or, the lower edges of the plurality of branches 1211 may exceed the edge of the bottom surface 212 of the backing part 210.

In some embodiments of the present disclosure, by providing the plurality of branches separated by the first spacing region, the plurality of branches can completely cover the first side surface, thereby effectively reducing the wrinkles caused by the main board portion disposed on the curved top surface of the backing part, saving space, and improving the circuit reliability.

In some embodiments, the backing part 210 may further include a second side surface connected to the other side of the top surface 211. The second side surface may be disposed on an opposite side (e.g., a side facing the inside of paper in FIG. 7) of the first side surface 213. The second side surface may have the same structure and functions as the first side surface 213. More descriptions regarding the second side surface may be found in the descriptions of the first side surface 213, which are not repeated here.

In some embodiments, as shown in FIG. 5, the flexible circuit board 100 may include a second connection portion 123. One end of the second connection portion 123 may be connected with the other side of the main board portion 110, and the other end of the second connection portion 123 may be configured to transmit electrical signals. In some embodiments, the second connection portion 123 may include a plurality of branches 1231. In some embodiments, two adjacent branches 1231 of the plurality of branches of the second connection portion 123 may be spaced by the first spacing region 1232. In some embodiments, the two adjacent branches 1231 of the plurality of branches of the second connection portion 123 may not be spaced by the first spacing region 1232. For example, side edges of the two adjacent branches 1231 of the plurality of branches may contact each other. In some embodiments, the plurality of branches 1231 of the second connection portion 123 may be disposed on the second side surface.

The second connection portion 123 may have the same or similar structure and functions as the first connection portion 121. More descriptions regarding the second connection portion 123 may be found in the descriptions of the first connection portion 121, which are not repeated here.

In some embodiments, a count of the plurality of branches 1211 of the first connection portion 121 may be the same as or different from a count of the plurality of branches 1231 of the second connection portion 123. In some embodiments, a first spacing distance between the two adjacent branches 1211 of the plurality of branches 1211 of the first connection portion 121 may be the same or different, and a second spacing distance between the two adjacent branches 1231 of the plurality of branches of the second connection portion 123 may be the same or different. In some embodiments, the first spacing distance between the two adjacent branches 1211 of the plurality of branches 1211 of the first connection portion 121 and the second spacing distance between the two adjacent branches 1231 of the plurality of branches of the second connection portion 123 may be the same or different. In some embodiments, the plurality of branches 1211 of the first connection portion 121 and the plurality of branches 1231 of the second connection portion 123 may have the same or different shapes and sizes.

In some embodiments, by providing the first connection portion and the second connecting portion, the flexible circuit board may be disposed on both sides of the first side surface and the second side surface of the backing part, such that the circuit can be fully arranged, the space can be saved, and stable signal transmission can be facilitated.

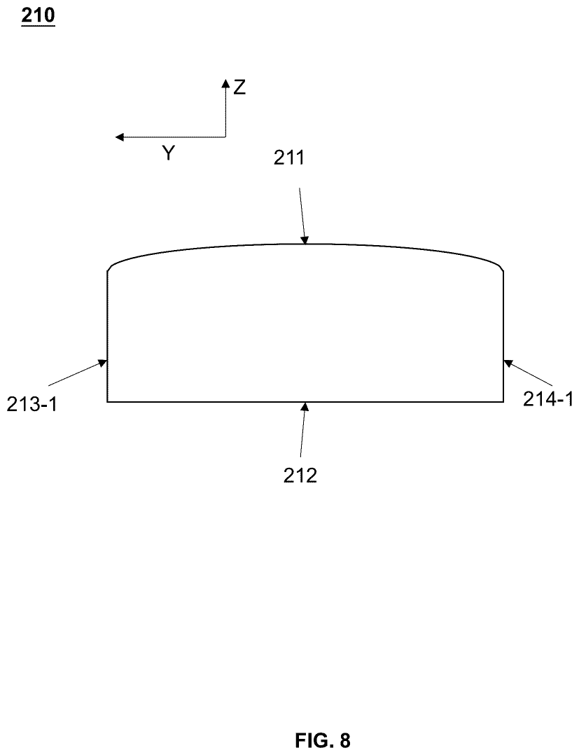

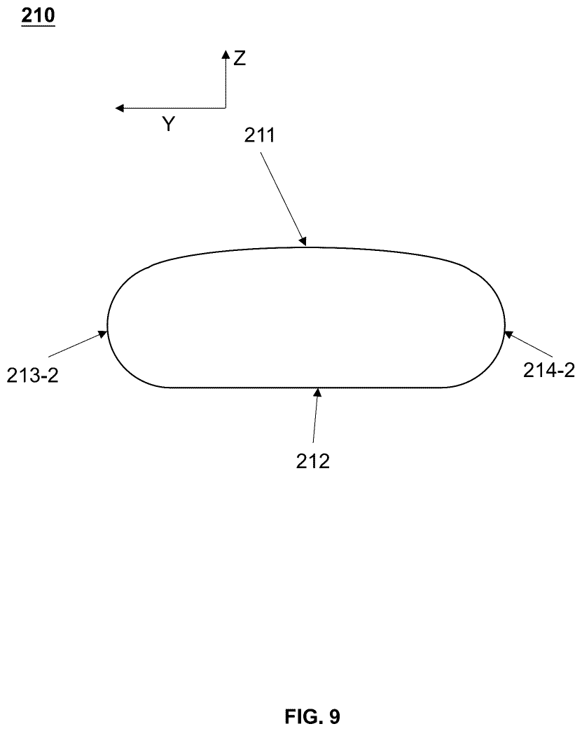

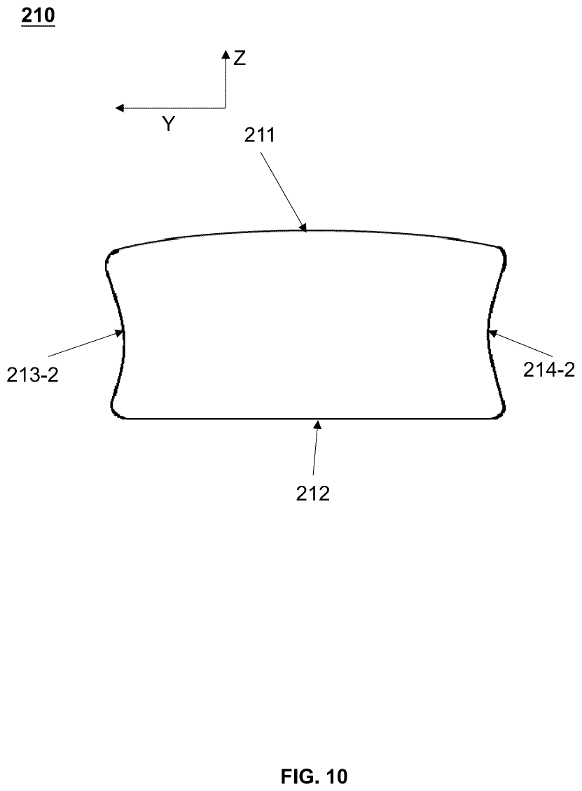

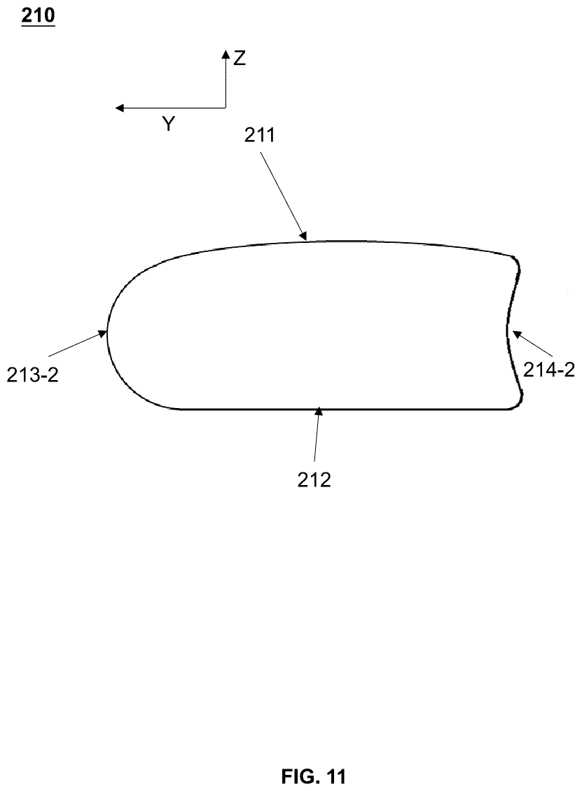

FIG. 8 is a cross-sectional view illustrating an exemplary backing part according to some embodiments of the present disclosure. FIG. 9 is a cross-sectional view illustrating an exemplary backing part according to some embodiments of the present disclosure. FIG. 10 is a cross-sectional view illustrating an exemplary backing part according to some embodiments of the present disclosure. FIG. 11 is a cross-sectional view illustrating an exemplary backing part according to some embodiments of the present disclosure. A cross section shown in FIGS. 8-11 may be parallel to a Y-Z plane.

In some embodiments, as shown in FIG. 8, a first side surface 213-1 may be straight planar. In some embodiments, as shown in FIG. 8, a second side surface 214-1 may be straight planar. In some embodiments, as shown in FIG. 8, both the first side surface 213-1 and the second side surface 214-1 may be straight planar.

In some embodiments, as shown in FIG. 9, a first side surface 213-2 may be outward convexly curved. Alternatively, as shown in FIG. 9, a second side surface 214-2 may be outward convexly curved. In some embodiments, as shown in FIG. 9, both the first side surface 213-2 and the second side surface 214-2 may be outward convexly curved.

In some embodiments, as shown in FIG. 10, the first side surface 213-2 may be inward concavely curved. In some embodiments, as shown in FIG. 10, the second side surface 214-2 may be inward concavely curved. In some embodiments, as shown in FIG. 10, the first side surface 213-2 and the second side surface 214-2 may both be inward concavely curved.

In some embodiments, as shown in FIG. 11, the first side surface 213-2 may be outward convexly curved. In some embodiments, as shown in FIG. 11, the second side surface 214-2 may be inward concavely curved. In some embodiments, the first side surface 213-2 may be inward concavely curved, and the second side surface 214-2 may be outward convexly curved.

By setting the first side surface and the second side surface to be curved, the bending angles of the first connection portion and the second connection portion being disposed on the first side surface and the second side surface can be reduced, thereby increasing the contact area and making the fit closer to reduce wrinkles.

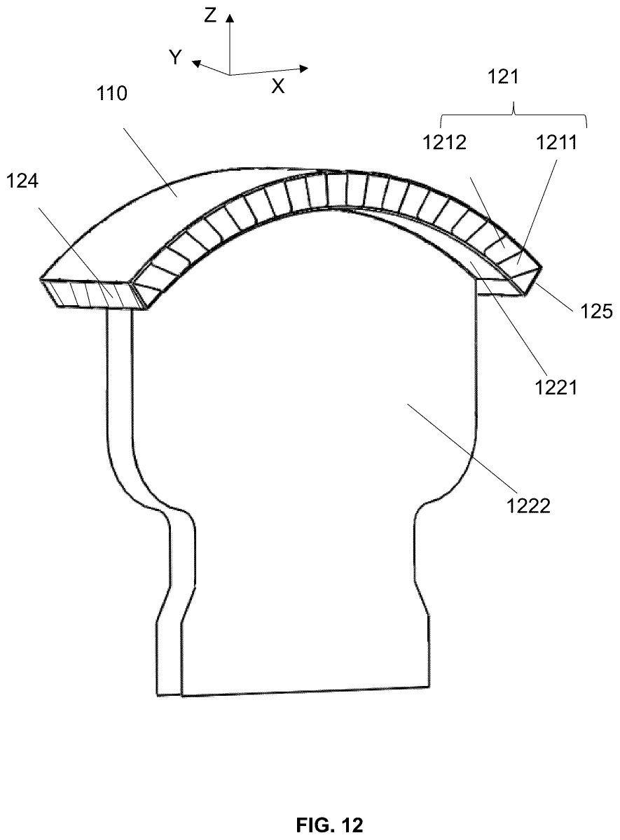

FIG. 12 is a structural diagram illustrating an exemplary ultrasonic probe according to some embodiments of the present disclosure.

In some embodiments, as shown in FIG. 7, the backing part 210 may further include a third side surface 215 and a fourth side surface 216. The first side surface 213 may be opposite to the second side surface, and the third side surface 215 may be opposite to the fourth side surface 216. In some embodiments, similar to the first side surface 213 and the second side surface, the third side surface 215 and the fourth side surface 216 may be straight planar or curved.

In some embodiments, as shown in FIG. 12, the flexible circuit board 100 may further include a third connection portion 124 and a fourth connection portion 125. The third connection portion 124 may be disposed on the third side surface 215, and the fourth connection portion 125 may be disposed on the fourth side surface 216.

In some embodiments, similar to the first connection portion 121, the third connection portion 124 and the fourth connection portion 125 may both include a plurality of branches, respectively. The plurality of branches of the third connection portion 124 and the fourth connection portion 125 may be the same or similar to the plurality of branches 1211 of the first connection portion 121. The plurality of branches of the third connection portion 124 and the fourth connection portion may be found in the descriptions of the plurality of branches 1211 of the first connection portion 121, which are not be repeated here. In some embodiments, the first connection portion 121, the second connection portion 123, the third connection portion 124, and the fourth connection portion 125 may be connected with each other, or may not be connected with each other.

By providing the first connection portion, the second connection portion, the third connection portion, and the fourth connection portion, the flexible circuit board can be disposed on the backing part in a sleeve manner, so as to adapt to the backing part within a certain size range, and improve the compatibility to the backing part of different sizes.

In some embodiments, the flexible circuit board 100 may include the first connection structure 122. The other end of the first connection portion 121 may be connected with the first connection structure 122.

The first connection structure 122 may be configured to connect the main board portion 110 with other components of the ultrasonic probe. For example, the first connection structure 122 may be configured to transmit a signal between the main board portion 110 and a host (i.e., the host of the ultrasonic device). In some embodiments, when the ultrasonic probe is not assembled, the first connection structure 122 and the main board portion 110 may be disposed on the same plane; after the ultrasonic probe is assembled, the first connection structure 122 and the main board portion 110 may not be disposed on the same plane. In some embodiments, the first connection structure 122 may be a “golden finger”. The golden finger may include a plurality of conductive contacts (e.g., golden conductive contacts), and may be referred to as the “golden finger” because a surface of the golden finger is gold-plated and the conductive contacts are arranged like fingers.

In some embodiments, a connection between the first connection portion 121 and the first connection structure 122 may be configured as a curved transition connection to avoid stress concentration and reduce or eliminate the problem of line breakage that may occur after bending.

In some embodiments, a width of an end of the first connection portion 121 connected with the main board portion 110 may be greater than a width of an end of the first connection portion 121 connected with the first connection structure 122, such that the flexible circuit board 100 is well matched with the shape of the backing part 210.

For descriptions regarding the first connection structure 122 may be found in the descriptions below.

In some embodiments, the flexible circuit board 100 may include a second connection structure. The other end of the second connection portion 123 may be connected with the second connection structure. The second connection structure may have the same or similar structure and functions as the first connection structure 122. More descriptions regarding the second connection structure may be found in the descriptions of the first connection structure 122.

In some embodiments, as shown in FIGS. 7-9, the backing part 210 may further include a bottom surface 212. In some embodiments, the bottom surface 212 of the backing part 210 may be a plane or a curved surface. In some embodiments, the top surface 211 and the bottom surface 212 of the backing part 210 may be a curved surface, respectively. The bottom surface 212 may be considered as an inner curved surface which may be concentric with an outer curved surface (i.e., the top surface 211) or may not be concentric with the outer curved surface (i.e., the top surface 211). In some embodiments, an edge length of the top surface 211 and an edge length of the bottom surface 212 of the backing part 210 may be equal or unequal. For example, the edge length of the top surface 211 may be greater than the edge length of the bottom surface 212 of the backing part 210. In some embodiments, a distance between the top surface 211 and the bottom surface 212 of the backing part 210 may be uniform or non-uniform. In some embodiments, the first side surface 213 of the backing part 210 may be connected between the top surface 211 and the bottom surface 212, and the upper and lower edges of the first side surface 213 may be connected with the edge of the top surface 211 and the edge of the bottom surface 212, respectively. In some embodiments, the first side surface 213 and the bottom surface 212 may be at a certain angle, such as 90° or other angles. In some embodiments, the first side surface 213 and the bottom surface 212 may be smoothly connected. In some embodiments, the first side surface 213 and the bottom surface 212 may be unsmoothly connected, i.e., a connection between the first side surface 213 and the bottom surface 212 may include a ridge.

In some embodiments, as shown in FIG. 5, FIG. 6, and FIG. 12, the first connection structure 122 may include a secondary board portion 1221 and an adapter portion 1222. The secondary board portion 1221 refers to a transition region disposed between the first connection portion 121 and the adapter portion 1222. In some embodiments, the secondary board portion 1221 may be considered as a portion of the first connection portion 121 or a portion of the adapter portion 1222, i.e., the secondary board portion 1221 and the first connection portion 121 (or the adapter portion 1222) may be configured as an integral piece.

In some embodiments, the first connection portion 121 may be connected between the main plate portion 110 and the secondary board portion 1221, and the secondary board portion 1221 may be connected between the first connection portion 121 and the adapter portion 1222. In some embodiments, the secondary board portion 1221 may be disposed on the bottom surface 212 of the backing part 210. In some embodiments, the first connection portion 121 may include a plurality of branches 1211. Each of the plurality of branches 1211 may be connected to the adapter portion 1222 through the secondary board portion 1221.

The secondary board portion of some embodiments of the present disclosure can be disposed on the bottom surface, such that a portion of the structure of the flexible circuit board can be disposed on the bottom of the backing part, and the flexible circuit board can better adapt to the shape of the backing part, thereby further preventing wrinkles, and saving space inside the ultrasonic probe.

In some embodiments, the bottom surface 212 of the backing part 210 may be curved, and a heat dissipation structure may be disposed on the bottom surface 212 of the backing part 210. A certain space may be formed under the curved bottom surface 212, and the heat dissipation structure may be mounted in the space formed under the bottom surface 212, so as to make full use of the space and achieve an effect of heat dissipation for the flexible circuit board 100. In some embodiments, other functional structures may be disposed on the bottom surface 212 as needed, such as a sound absorbing structure, a support structure, etc., to improve space utilization.

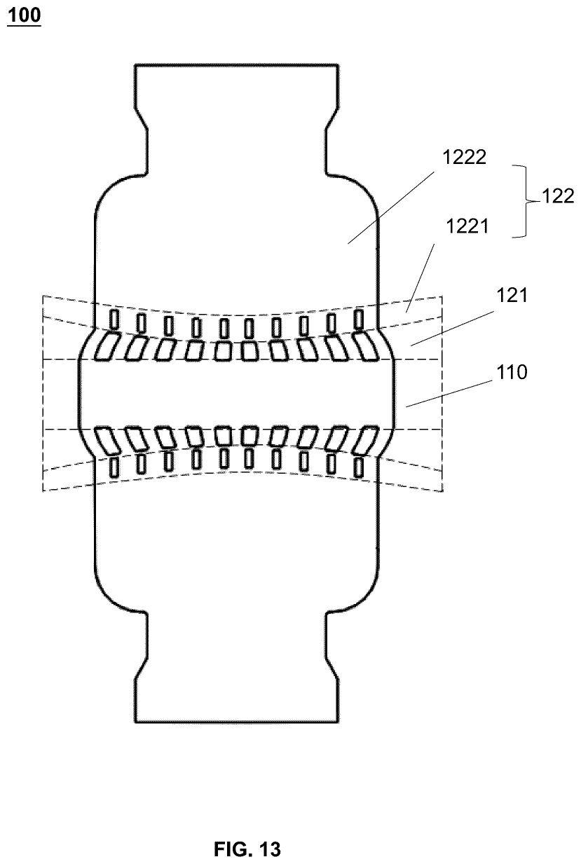

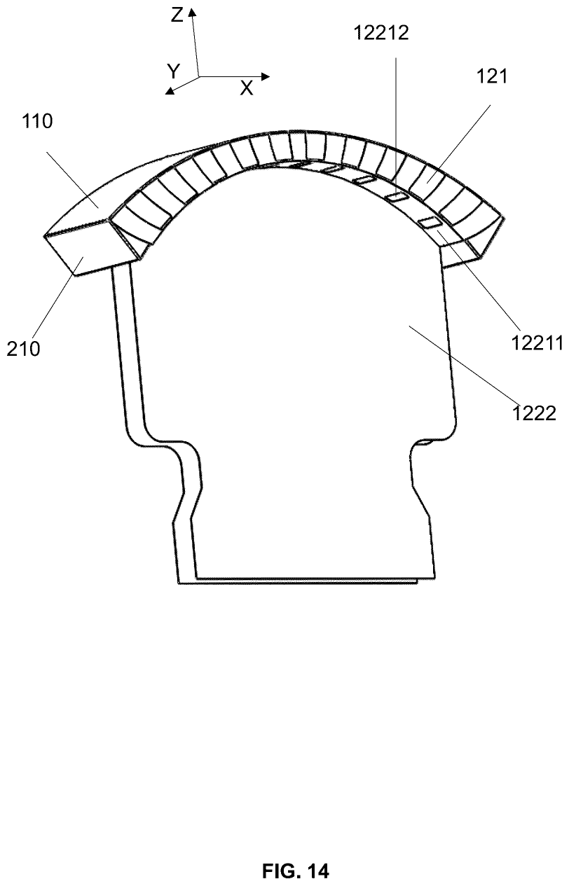

FIG. 13 is a structural diagram illustrating an exemplary flexible circuit board according to some embodiments of the present disclosure. FIG. 14 is a structural diagram illustrating an exemplary ultrasonic probe according to some embodiments of the present disclosure.

In some embodiments, as shown in FIGS. 13-14, the secondary board portion 1221 may include a plurality of branches 12211.

Two adjacent branches 12211 of the plurality of branches 12211 of the secondary board portion 1221 may be spaced by a second spacing region 12212. In some embodiments, the two adjacent branches 12211 of the plurality of branches 12211 of the secondary board portion 1221 may not be spaced by the second spacing region 12212. For example, side edges of the two adjacent branches 12211 of the plurality of branches 12211 may contact each other. The second spacing region 12212 may include a second hollow region. The second hollow region may be a hollow through hole disposed in the flexible circuit board 100. The second hollow region may include, but is not limited to, a rectangle, an ellipse, and other shapes.

In some embodiments, the second spacing region 12212 may include a second flexible region. The second flexible region refers to a region in the flexible circuit board 100 that is more flexible than other regions. In some embodiments, the second flexible region may have the same degree of flexibility as the first flexible region. In some embodiments, a rigidity of the second flexible region may be less than a rigidity of each of the plurality of branches 12211 of the secondary board portion 1221. The adjacent branches of the plurality of branches 12211 may be connected by a more flexible material, which does not affect the circuit performance, wrinkles are not liable to be generated when the secondary board portion is fit to the bottom surface of the backing part, and the process operation is simpler.

In some embodiments, the second flexible region may be elastic. By providing an elastic region between the adjacent branches of the plurality of branches, the backing part and the secondary board portion can be allowed to have a certain size change or tolerance to a certain extent, further improving the fit degree between the secondary board portion and the backing part.

In some embodiments, the plurality of branches 12211 of the secondary board portion 1221 and the plurality of branches 1211 of the first connection portion 121 are not limited, as long as the circuits in the secondary board portion 1221 and the first connection portion 121 can be well arranged and reliable electrical connection can be achieved between the secondary board portion 1221 and the first connection portion 121. For example, a count of the plurality of branches 12211 of the secondary board portion 1221 and a count of the plurality of branches 1211 of the first connection portion 121 may be the same or different. For example, the plurality of branches 12211 of the secondary board portion 1221 and the plurality of branches 1211 of the first connection portion 121 may have the same or different shapes and sizes. In some embodiments, one end of each of the plurality of branches 12211 of the secondary board portion 1221 may be connected with the other end of each of the plurality of branches 1211 of the first connection portion 121, and the other end of each of the plurality of branches 12211 of the secondary board portion 1221 may be connected with the adapter portion 1222.

In some embodiments, one or both ends of the second spacing region 12212 between the plurality of branches 12211 may be provided with an arced chamfer. The arced chamfer may reduce the stress concentration after the flexible circuit board is bent, thereby preventing breakage of the flexible circuit board during use.

In some embodiments of the present disclosure, by providing the hollow second spacing region in the secondary board portion, the surface tension of the flexible circuit board when the flexible circuit board is bent can be weakened, which helps to eliminate the wrinkles and makes it easier to bend.

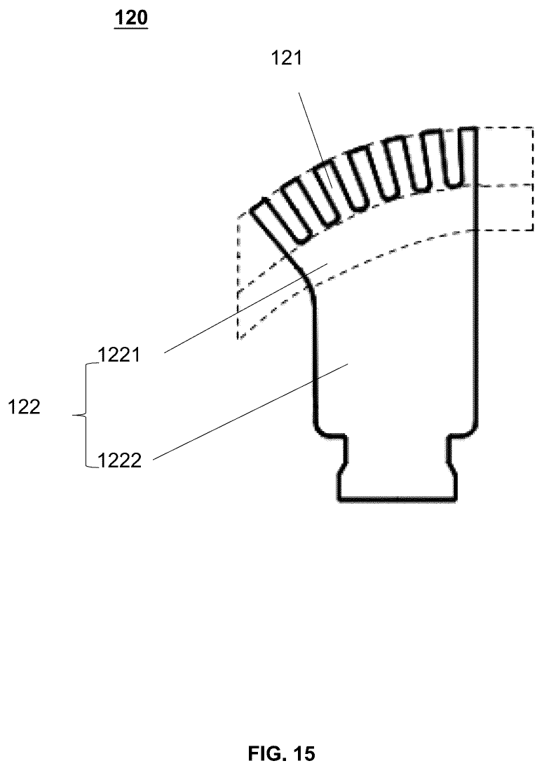

FIG. 15 is a structural diagram illustrating an exemplary adapter board according to some embodiments of the present disclosure.

In some embodiments, as shown in FIG. 15, the first connection portion 121 and the first connection structure 122 may form the adapter board 120. The adapter board 120 may be configured to implement signal transmission between the main board portion 110 and a host. In some embodiments, the first connection portion 121 and the first connection structure 122 may form the adapter board 120 by bonding, welding, or other connection modes. In some embodiments, the first connection portion 121 and the first connection structure 122 may form the adapter board 120 by integral molding. In some embodiments, the ultrasonic probe may include at least two adapter boards 120.

In some embodiments, the adapter board 120 may be relatively independent from the main board portion 110. A top end of the adapter board 120 may be connected with the main board portion 110 by, for example, binding, welding, connector connection, or other feasible connection modes, thereby simplifying the mounting process. A bottom end of the adapter board 120 may be connected with the host by, for example, plugging, snap-fit, or other feasible connection modes. In some embodiments, the adapter board 120 may be integrally formed with the main board portion 110.

In some embodiments, the top end of the adapter board 120 may be curved. If the ultrasonic probe is a convex array probe, the curved top end of the adapter board 120 may match a shape of the main board portion 110 and the backing part 210 to realize a stable connection with the main board portion 110, which helps signal transmission and effectively reduces wrinkles generated by the flexible circuit board 100, thereby preventing the flexible circuit board 100 from occupying too much space.

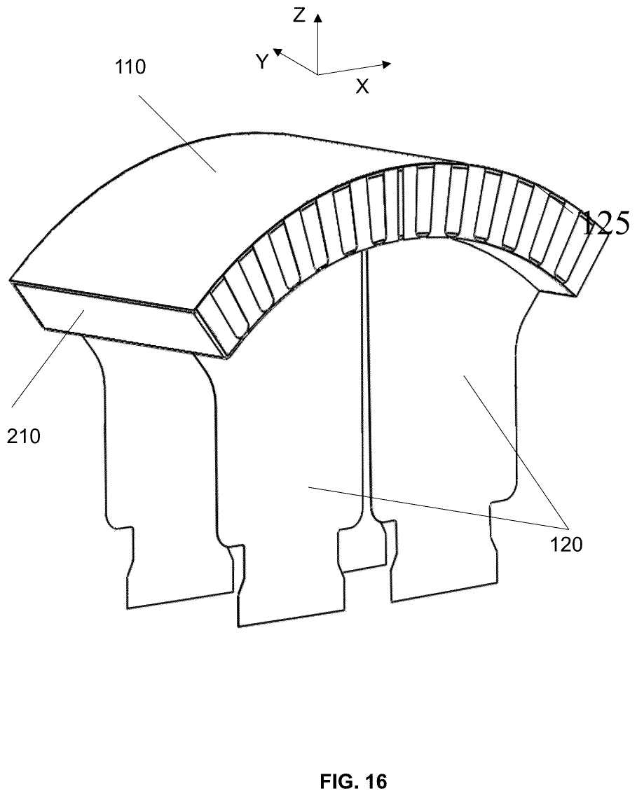

FIG. 16 is a structural diagram illustrating an exemplary ultrasonic probe according to some embodiments of the present disclosure.

In some embodiments, at least two adapter boards 120 may be provided. The at least two adapter boards 120 may be respectively connected with any two sides of the main board portion 110 to save space and improve the reliability of electrical connection, avoiding dense arrangement of circuits caused by arranging the circuits on a single side of the main board portion 110, and reducing the defective rate of plugging and unplugging.

In some embodiments, a plurality of adapter boards 120 may be provided, such as four adapter boards 120 shown in FIG. 16. A count of the adapter board 120 is not limited as long as the structure allows and a good signal transmission function can be achieved.

In some embodiments, as shown in FIG. 5, the main board portion 110 may have a first pre-fold a disposed between the main board portion 110 and the first connection portion 121. A position of the first pre-fold a may correspond to a position of a ridge between the top surface and the first side surface after the flexible circuit board 100 is disposed on the backing part 210. The position may be a connection between the top surface and the first side surface. In some embodiments, the flexible circuit board 100 may be processed such that the first pre-fold a is more easily bent at the position with respect to other positions of the flexible circuit board 100. For example, physical properties (e.g., stiffness) of the corresponding position may be changed through processing, such that the flexible circuit board 100 may be easier to bend at the position of the pre-fold. In some embodiments, the flexible circuit board 100 may be bent along the first pre-fold a. After being bent, the flexible circuit board 100 may present any angle within a range 0-180°, such as 90°, 110°, etc., with respect to both sides of the first pre-fold a.

In some embodiments, as shown in FIG. 5, a second pre-fold b may be disposed between the secondary board portion 1221 and the first connection portion 121. A position of the second pre-fold b may correspond to a position of a ridge between the first side surface and the bottom surface after the flexible circuit board 100 is disposed on the backing part 210. The position may be a connection between the first side surface and the bottom surface. In some embodiments, the flexible circuit board 100 may be processed such that the second pre-fold b is more easily bent at the position with respect to other positions of the flexible circuit board 100. In some embodiments, the flexible circuit board 100 may be bent along the second pre-fold b. After being bent, the flexible circuit board 100 may present any angle within a range of 0-180°, such as 90°, 110°, etc., with respect to both sides of the second pre-fold b. In some embodiments, a length of the first pre-fold a may be greater than a length of the second pre-fold b.

In some embodiments, as shown in FIG. 5, a third pre-fold c may be disposed between the secondary board portion 1221 and the adapter portion 1222. A position of the third pre-fold c may correspond to a position of a ridge between the bottom surface and a plane where the adapter portion 1222 is located after the flexible circuit board 100 is disposed on the backing part 210. The bottom surface and the plane where the adapter portion 1222 is located may be connected at an angle, such as 90°. Then, the bottom surface and the plane where the adapter portion 1222 is located may have a ridge, and the ridge may be at a position where the bottom surface and the plane where the adapter portion 1222 is located is connected. In some embodiments, the flexible circuit board 100 may be processed such that the third pre-fold c is more easily bent at the position with respect to other positions of the flexible circuit board 100. In some embodiments, the flexible circuit board 100 may be bent along the third pre-fold c. After being bent, the flexible circuit board 100 may present any angle within a range of 0-180°, such as 90°, 110°, etc., with respect to both sides of the third pre-fold c.

In some embodiments, the first pre-fold a, the second pre-fold b, and the third pre-fold c may have actual structures, or may be preset positions for folding rather than actual structures. When being bent, the flexible circuit board 100 may be folded along the first pre-fold a, the second pre-fold b, and/or the third pre-fold c. The first pre-fold a, the second pre-fold b, and the third pre-fold c may be provided to facilitate assembly personnel to assemble the flexible circuit board 100. In some embodiments, a pre-fold may be formed by bending before actual assembly, and then assembly may be performed according to the pre-fold to improve the assembly accuracy.

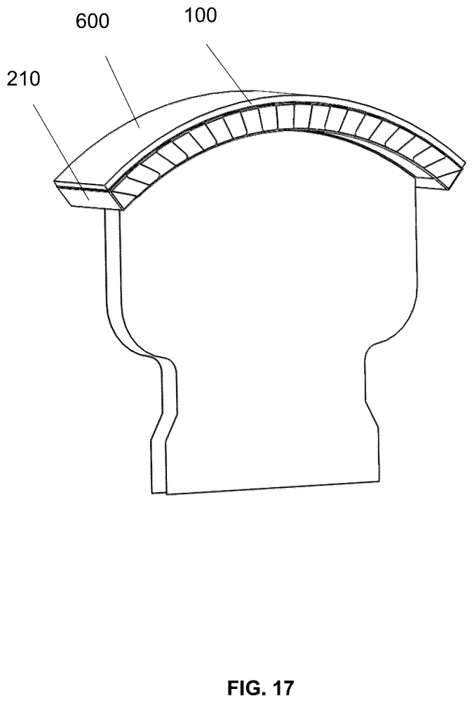

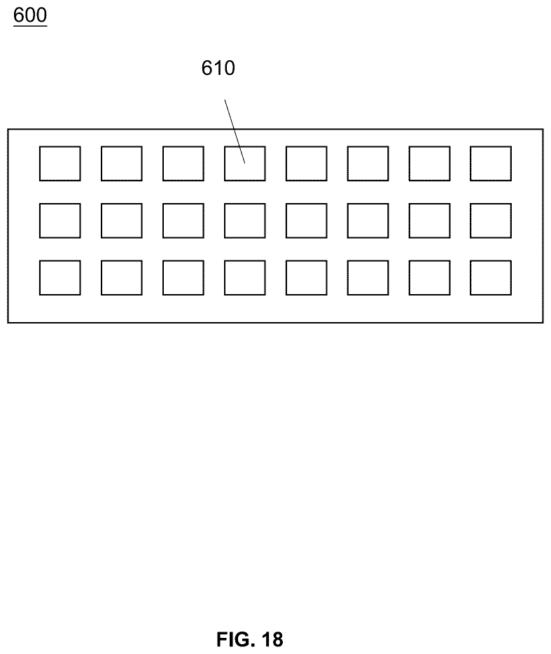

FIG. 17 is a structural diagram illustrating an exemplary connection between a piezoelectric layer and a flexible circuit board and a backing part according to some embodiments of the present disclosure. FIG. 18 is a structural diagram illustrating an exemplary piezoelectric layer according to some embodiments of the present disclosure.

In some embodiments, as shown in FIG. 17, the ultrasonic probe may further include a piezoelectric layer 600. A material of the piezoelectric layer 600 may include but is not limited to quartz crystal, barium titanate, lead zirconate titanate, etc. The flexible circuit board 100 may be disposed between the piezoelectric layer 600 and the backing part 210. The flexible circuit board 100 may be configured to supply power to the piezoelectric layer 600. The piezoelectric layer 600 may be configured to emit an acoustic wave signal based on a piezoelectric effect, and the acoustic wave may again cause the piezoelectric effect of the piezoelectric layer 600 after reflection to generate a signal. The flexible circuit board 100 may be electrically connected with the piezoelectric layer 600 to transmit the signal from the piezoelectric layer 600. The signal from the piezoelectric layer 600 may be received and processed by the host after being converted into an electrical signal.

In some embodiments, as shown in FIG. 18, the piezoelectric layer 600 may include a plurality of crystal units 610 arranged in an array. The plurality of crystal units 610 may correspond to the array elements described above. For example, the piezoelectric layer 600 may be an array of piezoelectric ceramic wafers.

In some embodiments, a first spacing distance between the two adjacent branches 1211 of the plurality of branches 1211 of the first connection portion 121 may be positively correlated with a third spacing distance between two adjacent crystal units 610 of the plurality of crystal units. In some embodiments, a second spacing distance between two adjacent branches 1231 of a plurality of branches 1231 of the second connection portion 123 may be positively correlated with the third spacing distance between the two adjacent crystal units 610 of the plurality of crystal units 610. That is, the larger the third spacing distance between the two adjacent crystal units 610 of the plurality of crystal units 610, the larger the first spacing distance between the two adjacent branches 1211 of the plurality of branches 1211 of the first connection portion 121 and the second spacing distance between the two adjacent branches of the plurality of branches of the second connection portion 123 may be set, so as to correspond to the array arrangement of the plurality of crystal units 610, and improve the rationality of the arrangement setting.

Some embodiments of the present disclosure further provide an ultrasonic device, such as an ultrasonic diagnostic instrument. The ultrasonic device may include the ultrasonic probe of one or more of the above embodiments. By using the ultrasonic probe of one or more of the above embodiments, the internal structure of the ultrasonic device is compact, the circuit connection is reliable, and the imaging quality effect is good.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or features may be combined as suitable in one or more embodiments of the present disclosure.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, numbers describing the number of ingredients and attributes are used. It should be understood that such numbers used for the description of the embodiments use the modifier “about”, “approximately”, or “substantially” in some examples. Unless otherwise stated, “about”, “approximately”, or “substantially” indicates that the number is allowed to vary by ±20%. Correspondingly, in some embodiments, the numerical parameters used in the description and claims are approximate values, and the approximate values may be changed according to the required features of individual embodiments. In some embodiments, the numerical parameters should consider the prescribed effective digits and adopt the method of general digit retention. Although the numerical ranges and parameters used to confirm the breadth of the range in some embodiments of the present disclosure are approximate values, in specific embodiments, settings of such numerical values are as accurate as possible within a feasible range.

For each patent, patent application, patent application publication, or other materials cited in the present disclosure, such as articles, books, specifications, publications, documents, or the like, the entire contents of which are hereby incorporated into the present disclosure as a reference. The application history documents that are inconsistent or conflict with the content of the present disclosure are excluded, and the documents that restrict the broadest scope of the claims of the present disclosure (currently or later attached to the present disclosure) are also excluded. It should be noted that if there is any inconsistency or conflict between the description, definition, and/or use of terms in the auxiliary materials of the present disclosure and the content of the present disclosure, the description, definition, and/or use of terms in the present disclosure is subject to the present disclosure.

Finally, it should be understood that the embodiments described in the present disclosure are only used to illustrate the principles of the embodiments of the present disclosure. Other variations may also fall within the scope of the present disclosure. Therefore, as an example and not a limitation, alternative configurations of the embodiments of the present disclosure may be regarded as consistent with the teaching of the present disclosure. Accordingly, the embodiments of the present disclosure are not limited to the embodiments introduced and described in the present disclosure explicitly.

What is claimed is:

1. An ultrasonic probe, comprising a backing part and a flexible circuit board, wherein:
the backing part includes a curved top surface and a first side surface connected to one side of the top surface;
the flexible circuit board includes a main board portion and a first connection portion, the main board portion being disposed on the top surface;
one end of the first connection portion is connected with one side of the main board portion, and the other end of the first connection portion is configured to transmit an electrical signal; and
the first connection portion includes a plurality of branches, the plurality of branches of the first connection portion being disposed on the first side surface.

2. The ultrasonic probe of claim 1, wherein:
the backing part further includes a second side surface connected to the other side of the top surface;
the flexible circuit board includes a second connection portion, one end of the second connection portion being connected with the other side of the main board portion, the other end of the second connection portion being configured to transmit the electrical signal;
the second connection portion includes a plurality of branches, the plurality of branches of the second connection portion being disposed on the second side surface.

3. The ultrasonic probe of claim 2, wherein the first side surface and/or the second side surface are outward convexly curved or inward concavely curved.

4. The ultrasonic probe of claim 2, wherein:
the backing part further includes a third side surface and a fourth side surface, the first side surface being opposite to the second side surface, the third side surface being opposite to the fourth side surface; and
the flexible circuit board further includes a third connection portion and a fourth connection portion.

5. The ultrasonic probe of claim 2, wherein:
a first count of the plurality of branches of the first connection portion and a second count of the plurality of branches of the second connection portion are the same or different;
a first spacing distance between two adjacent branches of the plurality of branches of the first connection portion and a second spacing distance between two adjacent branches of the plurality of branches of the second connection portion are the same or different.

6. The ultrasonic probe of claim 1, wherein two adjacent branches of the plurality of branches of the first connection portion are spaced by a first spacing region.

7. The ultrasonic probe of claim 6, wherein:
the first spacing region includes first hollow region; or
the first spacing region includes a first flexible region, a rigidity of the first flexible region being less than a rigidity of each of the plurality of branches of the first connection portion.

8. The ultrasonic probe of claim 7, wherein the first flexible region is elastic.

9. The ultrasonic probe of claim 6, wherein at least one end of the first spacing region is provided with an arced chamfer.

10. The ultrasonic probe of claim 6, further comprising a piezoelectric layer, wherein:
the flexible circuit board is disposed between the piezoelectric layer and the backing part, the flexible circuit board being configured to transmit a signal from the piezoelectric layer;
the piezoelectric layer includes a plurality of crystal units arranged in an array; and
a first spacing distance between the two adjacent branches of the plurality of branches of the first connection portion is positively correlated with a third spacing distance between two adjacent crystal units of the plurality of crystal units.

11. The ultrasonic probe of claim 1, wherein the flexible circuit board includes a first connection structure, and the other end of the first connection portion is connected with the first connection structure.

12. The ultrasonic probe of claim 11, wherein:
the backing part further includes a bottom surface, the first side surface being connected between the top surface and the bottom surface; and
the first connection structure includes a secondary board portion and an adapter portion, the first connection portion being connected between the main board portion and the secondary board portion, the secondary board portion being connected between the first connection portion and the adapter portion, and the secondary board portion being disposed on the bottom surface.

13. The ultrasonic probe of claim 12, wherein the secondary board portion includes a plurality of branches.

14. The ultrasonic probe of claim 13, wherein two adjacent branches of the plurality of branches of the secondary board portion are spaced by a second spacing region.

15. The ultrasonic probe of claim 14, wherein:
the second spacing region includes a second hollow region; or
the second spacing region includes a second flexible region, a rigidity of the second flexible region being less than a rigidity of each of the plurality of branches of the secondary board portion.

16. The ultrasonic probe of claim 15, wherein the second flexible region is elastic.

17. The ultrasonic probe of claim 11, wherein the first connection portion and the first connection structure form an adapter board, a top end of the adapter board being connected with the main board portion; the ultrasonic probe includes at least two adapter boards.

18. The ultrasonic probe of claim 17, wherein the top end of the adapter board is curved.

19. The ultrasonic probe of claim 12, wherein the bottom surface of the backing part is curved, and the bottom surface of the backing part is provided with a heat dissipation structure.

20. An ultrasonic device, comprising an ultrasonic probe, wherein:
the ultrasonic probe includes a backing part and a flexible circuit board;
the backing part includes a curved top surface and a first side surface connected to one side of the top surface;
the flexible circuit board includes a main board portion and a first connection portion, the main board portion being disposed on the top surface;
one end of the first connection portion is connected with one side of the main board portion, and the other end of the first connection portion is configured to transmit an electrical signal; and
the first connection portion includes a plurality of branches, the plurality of branches of the first connection portion being disposed on the first side surface.
